(19) United States
(12) Patent Application Publication
(10) Pub. No.: US 2013/0138103 A1
(43) Pub. Date: May 30, 2013

(54) ELECTROSURGICAL UNIT WITH MICRO/NANO STRUCTURE AND THE MANUFACTURING METHOD THEREOF

(71) Applicants: Chung-Wei CHENG, Miaoli County (TW); Cen-Ying LIN, Kaohsiung City (TW); Weng-Peng TSENG, Tainan City (TW); Keng-Liang OU, Kaohsiung City (TW); Pei-Wen PENG, Taipei City (TW)

(72) Inventors: Chung-Wei CHENG, Miaoli County (TW); Cen-Ying LIN, Kaohsiung City (TW); Weng-Peng TSENG, Tainan City (TW); Keng-Liang OU, Kaohsiung City (TW); Pei-Wen PENG, Taipei City (TW)

(73) Assignees: Taipei Medical University, Taipei City (TW); INDUSTRIAL TECHNOLOGY RESEARCH INSTITUTE, Hsin-Chu (TW)

(21) Appl. No.: 13/337,540

(22) Filed: Dec. 27, 2011

(30) Foreign Application Priority Data
Nov. 25, 2011 (TW) 100143427

(51) Int. Cl. A61B 18/14; B23K 26/00

(52) U.S. Cl. 606/45; 219/121.85

(57) ABSTRACT

An electrosurgical unit having micro/nano structure formed thereon and the manufacturing method thereof are disclosed, in which the electrosurgical unit is formed by the irradiation of a laser beam upon a blade so as to have a hybrid of micro/nano elements formed on the surface of the blade. The application of the hybrid of micro/nano elements on the surface of the blade has proven to be a valuable asset not only in providing a non-stick surface and a good heat dissipation ability to the blade, but also in providing a electrosurgical blade that will not release any toxic material under high temperature.

### TECHNICAL FIELD

The present disclosure relates to an electrosurgical unit with micro/nano structure and the manufacturing method thereof, and more particularly, to an electrosurgical blade with non-stick surface and good heat dissipation ability that is achieved by having micro/nano structure formed on the surface thereof.

### TECHNICAL BACKGROUND

Minimally invasive surgery, being any medical procedures that are performed through tiny incisions instead of one large opening, is becoming more and more common in hospitals, because the incisions are small for enabling patients to have quicker recovery times and less discomfort than with conventional surgery—all with the same benefits.

Generally, it is common to have an electrosurgical unit to be used in a minimally invasive surgical procedure. It is known that the electrosurgical unit is a device for applying a high-frequency electric current to biological tissue as a means to cut, coagulate, desiccate, or fulgurate tissue. Electric current is a flow of electric charge through a conductive medium in a closed circuit. Consequently, in monopolar configuration, the required closed circuit is formed by attaching a patient to a return electrode so as to enable electric current to flow from an active electrode, through the a target tissue to the return electrode, and then back to an electrosurgical generator. On the other hand, in bipolar configuration, the voltage is applied to the patient using a pair of similarly-sized electrodes, and when a piece of tissue is held by the pair of electrodes, a high frequency electric current flows from one to the other electrode, heating the intervening tissue.

Clinically, it had been found that when a target tissue is being heated in an electrosurgical procedure, there can be eschar generated and built up on the electrosurgical blade of the electrosurgical unit, and thus adversely affecting the performance of the electrosurgical blade. Therefore, the electrosurgical blade may require to be cleaned frequently in one electrosurgical procedure, resulting that the operation time is prolonged and the probability of damaging the electrosurgical blade in a cleaning process is increased. Moreover, stray electrical currents emanating from the electrosurgical blade can inadvertently burn non-targeted tissues beyond the surgeon's limited field of vision, regardless of the surgeon's skill and judgment, not to mention that the eschar build-up during the electrosurgical procedure may adversely affect healing and may limit the successful outcome of the surgery. In addition, there can be fluorine acid emission and smoke being generated in the electrosurgical procedure that at the very least it is obnoxious to the staff as a respiratory irritant, but at worst, there is some question to the viability of viruses carried away in this smoke, even for inducing lung cancer.

Accordingly, there are already many studies focusing on the improvement of conventional electrosurgical units for overcoming the aforesaid shortcomings. One of which is a study disclosed in U.S. Pat. No. 4,333,467, which is a nonstick conductive coating made of an organic material and adapted to be coated on the surface of an electrosurgical blade.

Another such study is a fluorine-doped diamond-like coatings disclosed in U.S. Pat. No. 6,468,642, which provide a method of making a substrate coated with a fluorine-doped diamond-like coating. Using which, an electrosurgical blade can be coated by a layer of Teflon for providing the same with a non-stick surface, while allowing a layer of diamond film to be deposited outside the Teflon layer for improving the hardness of the blade and also the durability of the same.

Another such study is a method and apparatus for providing a conductive, amorphous non-stick coating disclosed in U.S. Pat. No. 6,270,831, in which a conductive, non-stick coating is provided using a ceramic material and also a manufacturing process is provide for producing a coating of titanium nitride on a substrate. The coating, that is made of a nonstick conductive material, can also be applied as a conformal coating on a variety of substrate shapes, depending upon the application. The coating is bio-compatible and can be applied to a variety of medical devices.

Another such study is an application and utilization of a water-soluble polymer on a surface disclosed in U.S. Pat. No. 6,783,525, in which methods, systems, and devices for applying and utilizing a water-soluble polymer on a surface to provide desirable properties to the surface. In a first embodiment, the water-soluble polymer at least partially fills one or more pores of a fluoropolymer or a porous metal, whereas the surface can be an electrosurgical electrode tip and thus the desirable properties may include the ability to attract water that assists in cooling and/or lubricating the tip, to create a low shear, sacrificial layer that protects and enhances the tip, to supply a radical scavenger or inhibitor that reduces damage at the tip.

Another such study is an application and utilization of a hybrid material in a surface coating of an electrosurgical instrument disclosed in U.S. Pat. No. 6,951,559, in which a hybrid material is used for forming a coating on the surface of an electrosurgical instrument whereas the hybrid material may be the combination of a fluoropolymer, a water-soluble polymer, catalytic particles that may be activated or other materials that enhance the properties, characteristics and/or attributes of the coated surface. By the presence of the hybrid material coating layer, the electrosurgical instrument is provided with a high temperature stability that withstands the temperatures of electrosurgical procedure and a flexibility to increase the durability of the electrosurgical instrument.

Furthermore, in U.S. Pat. No. 7,867,225 and U.S. Pat. No. 7,867,226, an electrosurgical instrument with needle electrode is provided, in which a conductive element is formed as a needle that is surrounded by an insulation layer except at a conductor tip portion of the conductive element, while allowing the conductor tip portion and insulation layer each to have unique geometric shapes and composition of the parts to reduce or eliminate the production of smoke and eschar and reduce tissue damage.

Moreover, in TW Pat. No. M322806, there is a surgical knife with nano-diamond coating being disclosed, which is substantially a blade coated with a layer of nanometer diamond film while allowing a core layer or a strength middle layer to be sandwiched between the blade and the nanometer diamond film, whereas the nanometer diamond film can be a diamond film or diamond-like carbon film.

Similarly, in CN2481292, a high-frequency (HF) nano-diamond film operation knife is disclosed, which is substantially a blade having a layer of nano-diamond film depositing on the surface thereof.

Conclusively, in all the aforesaid means, the performance improvement of an electrosurgical blade, including non-stick effect, heat dissipation and hardness enhancement, is achieved by a film of Teflon that is coated or deposited on the surface of the electrosurgical blade. However, when the electrosurgical blade with Teflon film is being heated to about 400° C., the Teflon film will be burned and thus release a toxic fume including toxic fluorine ions that can pose a serious health risk.

In some of the aforesaid studies, such as TWM322806 and CN2481292, the nano-scaled film that is formed on the surface of an electrosurgical blade by coating or depositing, is used primarily for enhancing the hardness of the electrosurgical blade. However, it is known that a film that is formed using either a means of coating or a mean of deposition can only be a film with nano-scaled structures or a film with micro-scaled structures, but not a film formed with nano-scaled structures and mico-scaled structures simultaneously. Moreover, since in most case the blade and the film are not made of the same material that the film is usually formed on the surface of the blade by coating or depositing and is not a structure layer constructed directly from the surface the blade, the film not only will release toxic fume while being heated by the operating electrosurgical blade for a period of time, but also the film can wear away accordingly.

### TECHNICAL SUMMARY

The present disclosure relates to an electrosurgical unit having micro/nano structure formed thereon and the manufacturing method thereof, in which the electrosurgical unit is formed by the irradiating of a laser beam upon a blade so as to produce a hybrid of micro/nano elements on the surface of the blade precisely at its working area. The application of the hybrid micro/nano elements on the surface of the blade has proven to be a valuable asset in providing a non-stick surface and a good heat dissipation ability to the blade.

To achieve the above object, the present disclosure provides an electrosurgical unit with micro/nano structure, comprising:
a handle; and
a blade, arranged at an end of the handle, and having a micro/nano structure composed of a hybrid of micro/nano elements to be formed on the surface thereof by a means of laser direct structuring.

Moreover, to achieve the above object, the present disclosure provides a method for manufacturing electrosurgical unit with micro/nano structure, comprising the steps of:
providing an electrosurgical unit including a blade;
irradiating a laser beam upon the blade; and
using the irradiation of the laser beam to construct directly a micro/nano structure on the surface of the blade while allowing the micro/nano structure to be composed of a hybrid of micro/nano elements.

Further scope of applicability of the present application will become more apparent from the detailed description given hereinafter. However, it should be understood that the detailed description and specific examples, while indicating exemplary embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The present disclosure will become more fully understood from the detailed description given herein below and the accompanying drawings which are given by way of illustration only, and thus are not limitative of the present disclosure and wherein:

FIG. 1 is a schematic diagram showing an electrosurgical unit micro/nano structure according to an embodiment of the present disclosure.

FIG. 2 is a flow chart depicting the steps performed in a method for manufacturing electrosurgical unit with micro/nano structure according to an embodiment of the present disclosure.

FIG. 3 is enlarged view of an A-A cross section of FIG. 1

FIG. 4 is a schematic diagram showing a contact angle formed between a droplet and a blade of the present disclosure.

FIG. 5A, FIG. 5B and FIG. 5C show a same micro/nano structure that is being magnified by different magnifications, whereas the micro/nano structure is formed on the surface of an electrosurgical blade by the irradiation of a laser beam with 150 pulses.

FIG. 6 shows photos of micro/nano structure that are formed by the use of 200 laser pulses, 250 laser pulses, 300 laser pulses and 350 laser pulses in respective.

FIG. 7A, FIG. 7B and FIG. 7C are photos of different surface conditions of an electrosurgical blade that is being irradiated by laser beams of different pulses that are larger than 300 pulses.

FIG. 8A, FIG. 8B, FIG. 8C and FIG. 8D shows the cutting results comparing the performance of an electrosurgical blade with the micro/nano structure of the present disclosure with a conventional electrosurgical blade without the micro/nano structure.

FIG. 9 shows photos of a live rabbit whose skin is being engaged by an electrosurgical blade with the micro/nano structure of the present disclosure after 0 hour, 24 hours, 48 hours, 72 hours in respective.

### DESCRIPTION OF THE EXEMPLARY EMBODIMENTS

For your esteemed members of reviewing committee to further understand and recognize the fulfilled functions and structural characteristics of the disclosure, several exemplary embodiments cooperating with detailed description are presented as the follows.

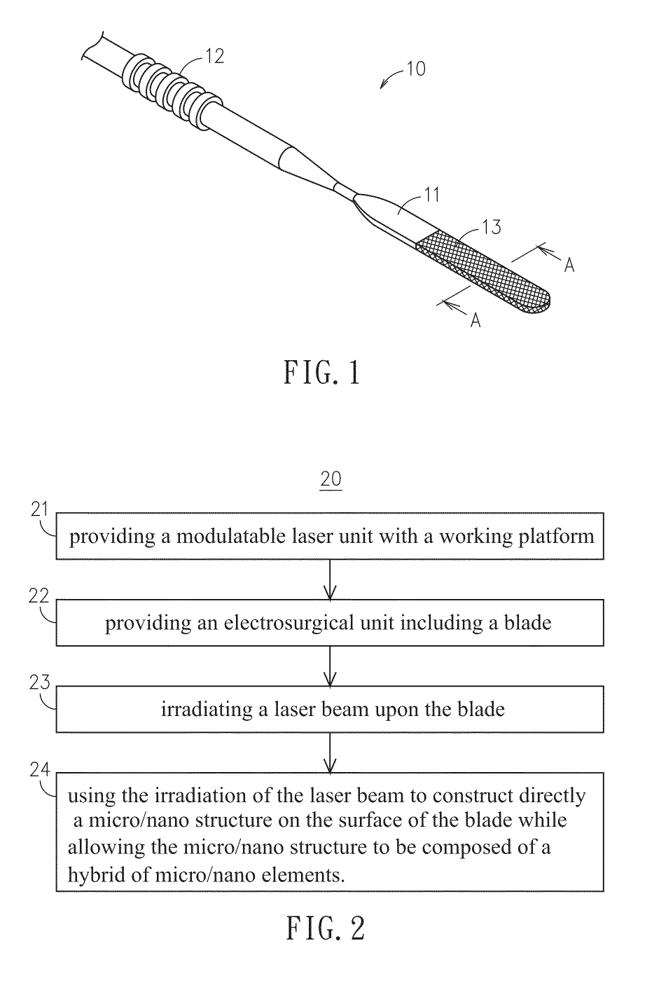

Please refer to FIG. 1, which is a schematic diagram showing an electrosurgical unit micro/nano structure according to an embodiment of the present disclosure. In FIG. 1, the electrosurgical unit 10 of the present disclosure can be an electrosurgical unit for minimally invasive surgery or an electrosurgical unit for conventional open surgery. Moreover, the electrosurgical unit 10 of the present disclosure can be constructed in a monopolar configuration or in a bipolar configuration, in which the monopolar electrosurgical unit can include a need-like, a plate-like, a ring-like, or a pen-like electrosurgical blade. As shown in FIG. 1, the electrosurgical unit 10 of the present disclosure comprises a blade 11 and a handle 12. The blade 11 that is arranged at an end of the handle 12 is made of a medial grade metal, such as a stainless steel. Moreover, since the blade 11 is conductive, the handle 12 should be made of an insulation material, by that a surgeon is able to use the blade 11 for performing an electrosurgical procedure upon a patient by holding the handle 12.

The electrosurgical unit 10 of the present disclosure is characterized in that: there is a micro/nano structure 13 formed on the surface of the blade 11 directly by the irradiation of a laser beam. That is, the micro/nano structure 13 is one-piece formed with the blade 11, and is not coated or deposited on the surface of the blade 11. Since the blade 13 is generally formed in a length longer than the cutting depth in electrosurgical procedures, only the front of the blade 11 is formed with the micro/nano structure 13 in this embodiment. Therefore, the cost and labor for forming the micro/nano structure 13 can be saved. Nevertheless, the micro/nano structure 13 can be formed on the blade 11 at areas other than the front, i.e. the micro/nano structure 13 can be formed on the blade 11 at any area as actually required. In another embodiment, the micro/nano structure 13 can be formed covering all the surface of the blade 11.

Please refer to FIG. 2, which is a flow chart depicting the steps performed in a method for manufacturing electrosurgical unit with micro/nano structure according to an embodiment of the present disclosure. As shown in FIG. 2, the manufacturing method comprises the following steps:
Step 21: providing a modulatable laser unit with a working platform;
Step 22: providing an electrosurgical unit including a blade;
Step 23: irradiating a laser beam upon the blade; and
Step 24: using the irradaation of the laser beam to construct directly a micro/nano structure on the surface of the blade while allowing the micro/nano structure to be composed of a hybrid of micro/nano elements.

In the step 21, the modulatable laser unit is further comprised of a focusing lens set with a numerical aperture value that is smaller than 0.25; and the working platform is configured with a fixture to be used for holding the electrosurgical unit that is to be formed with the micro/nano structure. In addition, the working platform is designed with X-Y-Z 3-axis adjustment capability in a manner that the adjustment in XY-axis is used for machining orientation adjustment, while the adjustment in Z-axis is used for adjusting the focal length of the laser beam. In this embodiment, the wavelength of the laser beam is ranged between 266 nm and 1064 nm, the energy of the laser beam is smaller than 0.26 W, the pulse width of the laser beam is ranged between 10 fs and 50 ps, and the frequency of the laser beam is ranged between 1 Hz and 1 MHz, the threshold value of the laser beam is ranged between 0.1 J/cm² and 8 J/cm², and the pulse number of the laser beam is smaller than 300 pulses. Nevertheless, all the aforesaid parameters of the laser beam including the wavelength, the threshold value, the pulse width and the frequency can be adjusted by a user according to actual requirement. Thereafter, after the parameters of the laser beam are set by a user according to an actual requirement and also the orientation and the positioning of the blade as well as the focus of the laser beam are adjusted, the steps 23 and 24 can be performed for forming the required micro/nano structure directly on the surface of the blade by the irradiation of the laser beam. It is noted that the laser beam can be modulated for enlarging the corresponding speckle by a hundred times, and thus the processing time can be reduced. In this embodiment, the laser beam is a femtosecond laser beam, however, it is not limited thereby.

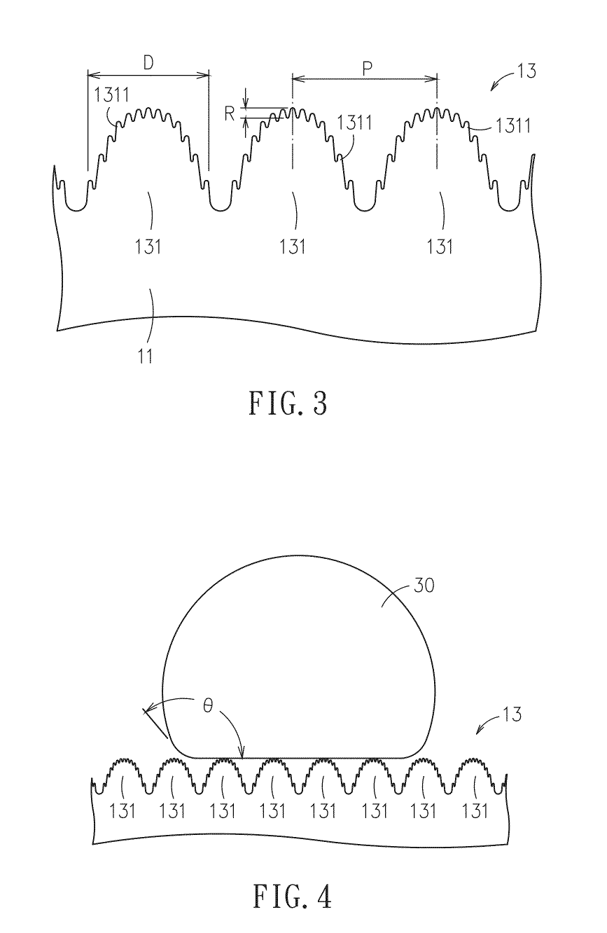

As shown in FIG. 3, the micro/nano structure 13 is formed directly on the blade 11, whereas the micro/nano structure 13 is composed of a hybrid of micro/nano elements 131. It is noted that in other embodiments of the present disclosure, there can be more than one type of micro/nano structures 13 being formed on the surface of the blade 11. In this embodiment, the hybrid of micro/nano elements 131 are spaced from one another by an interval P that is equal to or smaller than 9 μm; each of the hybrid micro/nano elements 131 is formed in a size D equal to or smaller than 5 μm.

In addition, each of the hybrid micro/nano elements 131 is formed with a periodic nanostructure 1311 in a size R equal to or smaller than 900 nm, and the periodic nanostructure 1311 is formed simultaneously with the formation of the micro/nano element 131. Moreover, each of the hybrid micro/nano elements 131 is formed with a surface roughness smaller than 90 nm. It is noted that the hybrid micro/nano elements 131 in this embodiment are actually being formed as irregular pillars of various sizes, so that hybrid micro/nano elements 131 can be an assembly of pillars of different shapes and sizes while allowing each of the hybrid micro/nano elements 131 to be formed nanoscale periodic strips on the surface thereof. It is noted that the aforesaid size for each of the hybrid micro/nano elements 131 is actually referred to the diameter or the width of the pillar-like micro/nano elements, and for those micro/nano elements that are pillars of irregular shapes, the size is referred to the maximum width of the irregular pillar. Moreover, the surface roughness is referred to the degree of concave that is resulted from the periodic nanostructure 1311 formed on the surface thereof, such as the nanoscale periodic strips. Consequently, by the steps depicted in FIG. 2, parameters of the laser beam including the wavelength, the threshold value, the pulse width and the frequency can be set by a user according to actual requirement so as to be used for forming the hybrid micro/nano elements 131 of corresponding interval, size and roughness on the blade 11, and collectively as one micro/nano structure 13. It is noted that the interval, size and roughness of the hybrid micro/nano elements 131 that are not described in the aforesaid embodiment are for illustration and thus they are not limited by those, and consequently, the interval, size and roughness of the hybrid micro/nano elements 131 that are not defined within the ranges mentioned in the aforesaid embodiment can still be used in the present disclosure.

Please refer to FIG. 4, which is a schematic diagram showing a contact angle formed between a droplet and a blade of the present disclosure. In FIG. 4, the droplet can be a drop of water, saline solution or blood. As shown in FIG. 4, a droplet 30 that is being dropped on the micro/nano structure 13 of the blade 11 is going to engaged with the hybrid micro/nano elements 131, causing a contact angle θ to be formed with respect to a level surface in a manner that the contact angle will be larger than 130 degrees when the droplet 30 is a drop of water or artificial body fluid, and the contact angle will be larger than 150 degrees when the droplet 30 is a drop of blood. Generally, if a surface is able to cause a contact angle that is larger than 90 degrees, the surface can be considered to have good hydrophobicity, i.e. the droplet 30 is not likely to permeate into the structure of the surface; on the other hand, if a surface is able to cause a contact angle that is smaller than 90 degrees, the surface can be considered to have good hydrophility, i.e. the droplet 30 can easily permeate into the structure of the surface. Since the blade 11 is formed with the micro/nano structure 13 for causing the contact angle θ of any droplet 30 on the surface of the blade 11 to be a larger-than-90-degree angle, the blade 11 is formed with good hydrophobicity so as to enhance the non-stick property of the blade; and moreover, as the hybrid micro/nano elements 131 are spaced from one another, the heat of the blade can be evenly dissipated for allowing less heat to be transmitted to the surrounding of the blade 11. In an experiment for comparing the performance of an electrosurgical blade with the micro/nano structure 13 of the present disclosure with a conventional electrosurgical blade without the micro/nano structure 13, the temperature of a tissue that is being cut by the conventional electrosurgical blade can achieve 145° C., while the temperature of another tissue that is being cut by the electrosurgical blade of the present disclosure is about 116° C. That is, the electrosurgical blade with the micro/nano structure 13 can cause the temperature to decent by more than 17.3%. Therefore, there will be less damage to the tissue that is being cut by the electrosurgical blade of the present disclosure as the heat of the blade can be evenly dissipated for allowing less heat to be transmitted to the surrounding of the blade 11.

As seen in the table provided below,

| | | Before | After | Weight |
|---|---|---|---|---|
| | Watt | surgery(g) | surgery(g) | difference(g) |
| conventional | 40 | 2.3280 | 2.3302 | 0.0022 |
| | 60 | 2.3200 | 2.3218 | 0.0018 |
| | 80 | 2.3132 | 2.3144 | 0.0012 |
| Present | 40 | 2.1150 | 2.1158 | 0.0008 |
| | 60 | 2.1225 | 2.1227 | 0.0002 |
| | 80 | 2.1283 | 2.1284 | 0.0001 |

while operating under three different power wattages, i.e. 40 W, 60 W and 80 W, the weight differences before surgery and after surgery using an electrosurgical blade of the present disclosure is much smaller than those using a conventional electrosurgical blade. Moreover, it is noted that the higher the power wattage is, the smaller the weight difference will be since there is less tissue stuck on blade. Thus, the electrosurgical blade with the micro/nano structure 13 of the present disclosure is proven to have good hydrophobicity so as to enhance the non-stick property of the blade.

It is emphasized that the micro/nano structure 13 is formed on the surface of the blade 11 by a one-step forming process using a laser beam, and the micro/nano structure is formed directly on the surface of the blade in a manner that the micro/nano structure 13 is one-piece formed with the blade 11. Conventionally, there are already many techniques that are provided for forming micro/nano structures, such as etching, plasma forming, and LIGA. However, using the prior-art etching methods, there will be a plurality of steps to be performed for forming the required micro/nano structure, and thus the micro/nano structure can not be formed in a one-step forming process. As for the prior-art plasma forming method, it is difficult to control the resulting shapes of the micro/nano structures that are being formed thereby. As for the prior-art LIGA method, there will be a plurality of steps to be performed for forming the required micro/nano structure in addition to the requirement for setting up an optic mask in the multi-step process, resulting that the manufacturing cost is increased and also the micro/nano structure is not being formed in a one-step forming process. As for those common precision machining and means using long-pulse laser, they can at best produce micro-scale structure, but are not capable of producing the required micro/nano structures. Therefore, the micro/nano structure of the present disclosure is formed by means of laser direct structuring with satisfactory structural precision.

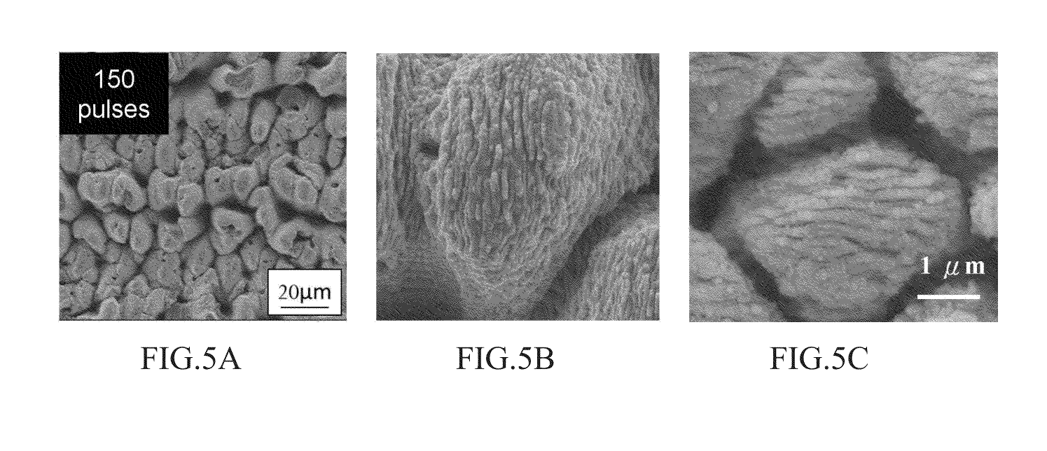

Using the manufacturing method provided in the present disclosure, an electrosurgical blade can be formed with a non-stick micro/nano structure, as shown in the sample photos of FIG. 5A, FIG. 5B and FIG. 5C. The FIG. 5A, FIG. 5B and FIG. 5C show a same micro/nano structure that is being magnified by different magnifications, whereas the micro/nano structure is formed on the surface of an electrosurgical blade by the irradiation of a laser beam with 150 pulses. As shown in FIG. 5A, FIG. 5B and FIG. 5C, the micro/nano structure is an assembly of a hybrid of pillar-like or particle-like micro/nano elements that are periodically distributed, and each of the plural micro/nano elements is formed with a periodic nanostructure in a manner that each of the hybrid pillar-like micro/nano elements is formed nanoscale periodic strips on the surface thereof.

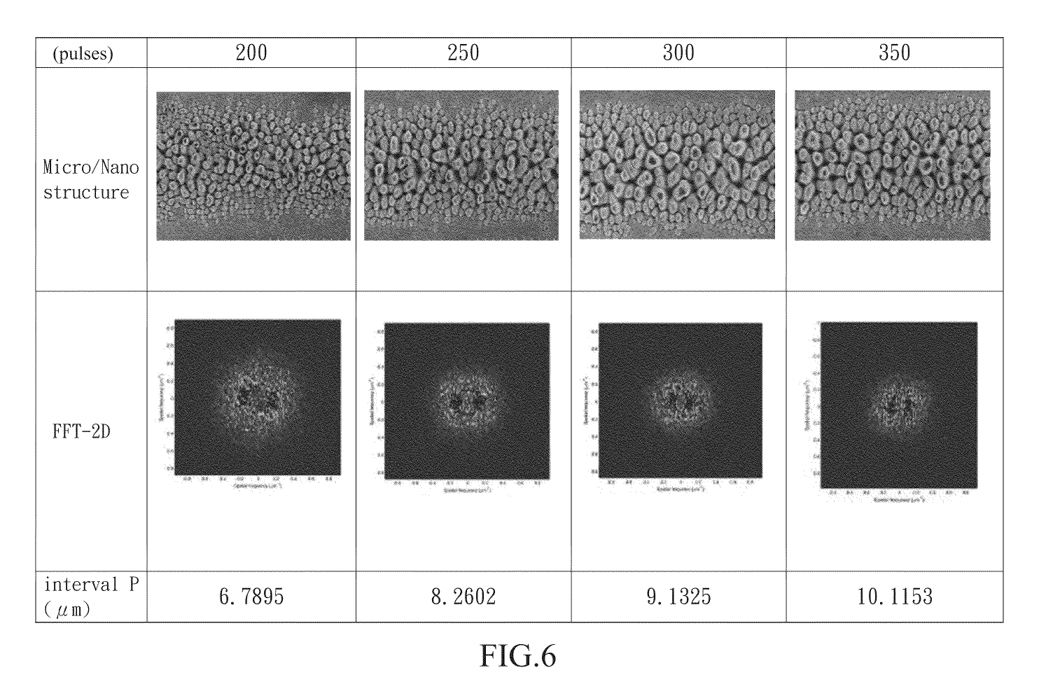

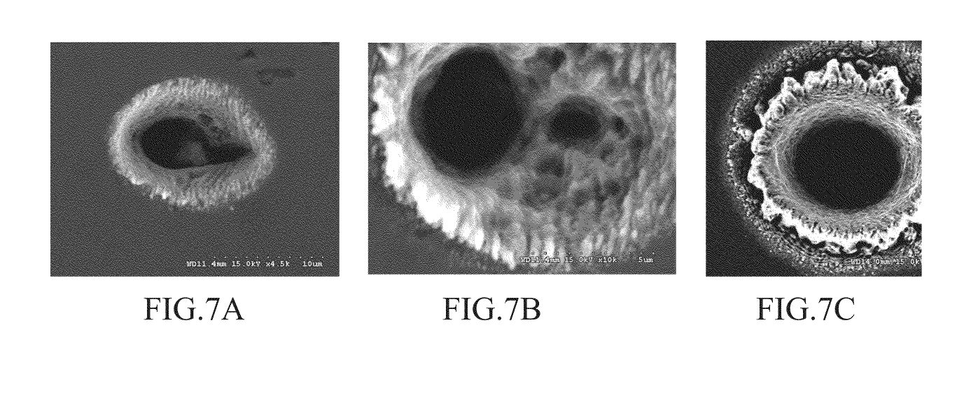

Regarding to the micro/nano structure that is formed on the surface of an electrosurgical blade of the present disclosure, the shape and size of the micro/nano structure that is being formed will be different depending on the pulse energy of the laser beam used. Please refer to FIG. 6, which shows photos of micro/nano structure that are formed by the use of 200 laser pulses, 250 laser pulses, 300 laser pulses and 350 laser pulses in respective. Experimentally, it is noted that when one micro/nano structure is formed by the use of a laser beam with a pulse number that is larger than 300 pulses, the resulting micro/nano elements in such micro/nano structure will be formed spaced from one another by an interval larger than 9 μm; and it is known that when the micro/nano elements are spaced from one another by an interval larger than 9 μm, the hydrophobicity of the resulting blade will be poor. Thus, the present disclosure proposes an experiment for producing the micro/nano structure by the use of 0.26 W laser beam with less than 300 pulses. Nevertheless, in a situation when the surface of an electrosurgical blade are being irradiated and processed by a laser beam with more than 300 pulses, it is possible that the surface of the electrosurgical blade can be damaged by the laser irradiation and thus no micro/nano structure can be formed on the surface of the electrosurgical blade. Please refer to the photos shown in FIG. 7A, FIG. 7B and FIG. 7C, which are different surface conditions of an electrosurgical blade that is being irradiated by laser beams of different pulses that are larger than 300 pulses. As shown in the FIG. 7A, FIG. 7B and FIG. 7C, there are holes being formed on the surface of the electrosurgical blade. However, if we irradiate the surface of an electrosurgical blade by a laser beams with larger-than 300 pulses, but with an energy smaller than 0.26 W, the surface of the electrosurgical blade will not be damaged and thus there will still be micro/nano structure formed, such laser setting can still be included in the spirit and scope of the present disclosure.

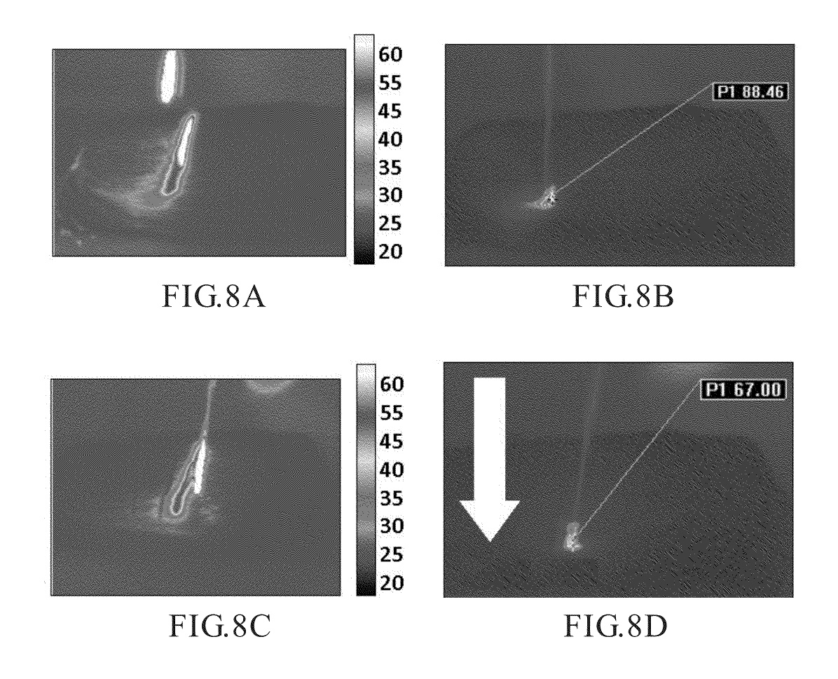

It is noted that the electrosurgical unit of the present invention is designed to achieve a better heat dissipating effect for enabling the heat of the blade to be evenly dissipated and thus for allowing less heat to be transmitted to the surrounding of the blade. Please refer to FIG. 8A, FIG. 8B, FIG. 8C and FIG. 8D, which shows the cutting results comparing the performance of an electrosurgical blade with the micro/nano structure of the present disclosure with a conventional electrosurgical blade without the micro/nano structure. In FIG. 8A and FIG. 8B, the temperature of a tissue that is being cut by the conventional electrosurgical blade of 80 W can achieve 88.46° C., while the temperature of another tissue that is being cut by the 80 W electrosurgical blade of the present disclosure is about 67° C., as shown in FIG. 8C and FIG. 8D. That is, the electrosurgical blade with the micro/nano structure can cause the temperature to decent by 21.46° C. Therefore, there will be less damage to the tissue that is being cut by the electrosurgical blade of the present disclosure as the heat of the blade can be evenly dissipated for allowing less heat to be transmitted to the surrounding of the blade, and also there will be less tissue or eschar stuck on the blade.

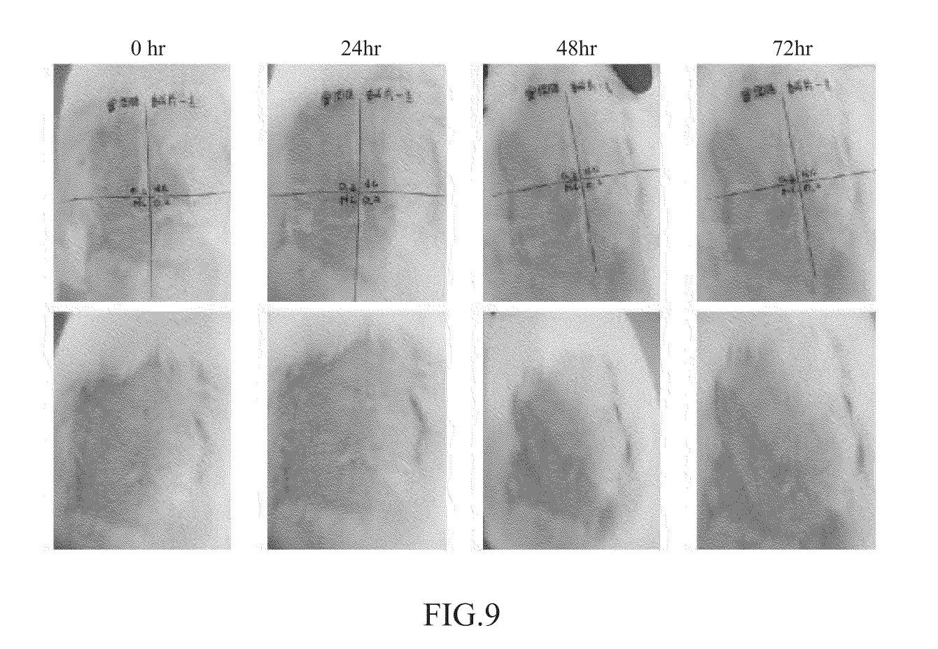

Please refer to FIG. 9, which are photos of a live rabbit whose skin is being engaged by an electrosurgical blade with the micro/nano structure of the present disclosure after 0 hour, 24 hours, 48 hours, 72 hours in respective. As show in the photos of FIG. 9, there is no irritation effect or inflammation shown on the rabbit.

To sum up, the present disclosure provides an electrosurgical unit having micro/nano structure formed thereon and the manufacturing method thereof, in which the electrosurgical unit is formed by the irradiation of a laser beam upon a blade so as to produce a hybrid of micro/nano elements on the surface of the blade that are periodically distributed. By controlling the energy of the laser beam irradiated on the surface of the electrosurgical blade, a hydrophobic micro/nano structure can be formed on the surface of the blade that is proven to be a valuable asset in providing a non-stick surface and a good heat dissipation ability to the blade. Moreover, by the application of the micro/nano structure, there will be less damage to the tissue that is being cut by the electrosurgical blade of the present disclosure as the heat of the blade can be evenly dissipated for allowing less heat to be transmitted to the surrounding of the blade, and also there will be no toxic fume being released for posing a serious health risk to the medical personal using the electrosurgical unit.

With respect to the above description then, it is to be realized that the optimum dimensional relationships for the parts of the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present disclosure.

What is claimed is:

1. An electrosurgical unit with micro/nano structure, comprising:
a handle; and
a blade, arranged at an end of the handle, and having a micro/nano structure composed of a hybrid of micro/nano elements.

2. The electrosurgical unit with micro/nano structure of claim 1, wherein the micro/nano structure is formed directly on the surface of the blade by the irradiation of a laser beam.

3. The electrosurgical unit with micro/nano structure of claim 2, wherein the wavelength of the laser beam is ranged between 266 nm and 1064 nm, the energy of the laser beam is smaller than 0.26 W, the pulse width of the laser beam is ranged between 10 fs and 50 ps, and the frequency of the laser beam is ranged between 1 Hz and 1 MHz.

4. The electrosurgical unit with micro/nano structure of claim 2, wherein the threshold value of the laser beam is ranged between 0.1 J/cm² and 8 J/cm².

5. The electrosurgical unit with micro/nano structure of claim 1, wherein the hybrid micro/nano elements are spaced from one another by an interval not larger than 9 μm.

6. The electrosurgical unit with micro/nano structure of claim 1, wherein each of the hybrid micro/nano elements is formed in a size not larger than 5 μm.

7. The electrosurgical unit with micro/nano structure of claim 1, wherein each of the hybrid micro/nano elements is formed with a surface roughness smaller than 90 nm.

8. The electrosurgical unit with micro/nano structure of claim 1, wherein each of the hybrid micro/nano elements is formed nanoscale periodic strips on the surface thereof.

9. The electrosurgical unit with micro/nano structure of claim 1, wherein each of the hybrid micro/nano elements is formed with a periodic nanostructure in a size not larger than 900 nm.

10. The electrosurgical unit with micro/nano structure of claim 1, wherein each of the hybrid micro/nano elements is formed for enabling the same with hydrophobicity so as to enhance the non-stick property of the blade; and the hybrid micro/nano elements are spaced from one another for improving the heat dissipating effect on the blade.

11. A method for manufacturing electrosurgical unit with micro/nano structure, comprising the steps of:
providing a modulatable laser unit with a working platform;
providing an electrosurgical unit including a blade;
irradiating a laser beam upon the blade; and
using the irradiation of the laser beam to construct directly a micro/nano structure on the surface of the blade while allowing the micro/nano structure to be composed of a hybrid of micro/nano elements.

12. The manufacturing method of claim 11, wherein the laser beam is a picosecond or femtosecond laser beam.

13. The manufacturing method of claim 11, wherein the modulatable laser unit is further comprised of a focusing lens set with a numerical aperture value that is smaller than 0.25; the working platform is configured with a fixture to be used for holding the electrosurgical unit that is to be formed with the micro/nano structure; the working platform is designed with X-Y-Z 3-axis adjustment capability.

14. The manufacturing method of claim 11, wherein the wavelength of the laser beam is ranged between 266 nm and 1064 nm, the energy of the laser beam is smaller than 0.26 W, the pulse width of the laser beam is ranged between 10 fs and 50 ps, and the frequency of the laser beam is ranged between 1 Hz and 1 MHz.

15. The manufacturing method of claim 11, wherein the hybrid micro/nano elements are spaced from one another by an interval not larger than 9 μm.

16. The manufacturing method of claim 11, wherein each of the hybrid micro/nano elements is formed in a size not larger than 5 μm.

17. The manufacturing method of claim 11, wherein each of the hybrid micro/nano elements is formed with a surface roughness smaller than 90 nm.

18. The manufacturing method of claim 11, wherein each of the hybrid micro/nano elements is formed with a periodic nanostructure in a size not larger than 900 nm; and the periodic nanostructure is formed simultaneously with the formation of the micro/nano element.

19. The manufacturing method of claim 11, wherein the threshold value of the laser beam is ranged between 0.1 J/cm² and 8 J/cm².
